(19) United States
(12) Patent Application Publication
(10) Pub. No.: US 2026/0272638 A1
(43) Pub. Date: Sep. 17, 2026

(54) ACCOMMODATING INTRAOCULAR LENS WITH FLUID CHANNELS

(71) Applicant: Adaptilens, Inc., Chestnut Hill, MA (US)

(72) Inventors: Liane Clamen, Chestnut Hill, MA (US); Brett Thomes, Chestnut Hill, MA (US); Dan Stanley, Chestnut Hill, MA (US); Jason Bryant, Chestnut Hill, MA (US); Mark Burchnall, Chestnut Hill, MA (US); Ben Ellis, Chestnut Hill, MA (US)

(73) Assignee: Adaptilens, Inc., Chestnut Hill, MA (US)

(21) Appl. No.: 19/568,559

(22) Filed: Mar. 16, 2026

Related U.S. Application Data
(60) Provisional application No. 63/773,135, filed on Mar. 17, 2025.

(51) Int. Cl. A61F 2/16

(52) U.S. Cl. CPC A61F 2/1624; A61F 2/1601

(57) ABSTRACT

A biomimetic accommodating intraocular lens (AIOL), configured to provide an amount of accommodation to allow a continuous range of vision, includes exterior channel features to control fluid flow around the periphery of the lens. The channel geometry may vary to improve fluid flow and may have a longitudinal profile of constant or varying depth. The number of channels may range from a single channel to numerous channels disposed around the circumference of the lens. The channel(s) may extend radially outward from the edges of the lens optic zones and transit the equator region of the lens body therebetween. The channel(s) allow the eye's aqueous humor to bathe the lens capsular bag, thereby decreasing the incidence of anterior and posterior capsule opacification. The channels allow for a smaller volume IOL to be compressed through a smaller limbal incision and for egress of OVD during the irrigation/aspiration step of cataract surgery.

### INCORPORATION BY REFERENCE

The present application claims the benefit of priority to U.S. Provisional Application No. 63/773,135 filed on Mar. 17, 2025, the entire disclosure of which is hereby incorporated by reference in its entirety.

### FIELD

Illustrative embodiments generally relate to intraocular lenses (IOL) and, more particularly, an accommodating intraocular lens (AIOL).

### BACKGROUND

The lens capsule, or the capsular bag, is a thin membrane around the eye's natural lens that holds the lens in a central position within the eye and helps give the lens its shape. The capsular bag comprises an anterior and posterior capsule. Attached to the periphery of the capsule are tiny string-like structures called zonules which attach to the ciliary muscle. The ciliary muscle plays a critical role in accommodation, the process that allows the eye to adjust focus for near, intermediate, and distance vision. For near vision, the ciliary muscle contracts and reduces the tension on the zonules. This allows the lens to become rounder, thus increasing its refractive power so the eye can focus on near objects. For distance vision, the ciliary muscle relaxes, increasing tension on the zonules and pulling the capsular bag taut. Thus, the lens flattens and has decreased refractive power, enabling the eye to focus on distant objects. As people reach about age 45 and older, the lens becomes stiffer and less able to change shape and power. With this age-related condition, called presbyopia, people must use glasses or contact lenses to see clearly at near and intermediate.

At about the age of 75, the natural lens becomes stiffer and cloudy and it is called a cataract. A cataract can be removed with cataract surgery during which an artificial intraocular lens or IOL is inserted into the lens capsule in place of the cloudy cataractous lens. Sometimes the capsule itself can become cloudy over time after surgery, a condition known as posterior capsule opacification (PCO).

An accommodating intraocular lens is a type of IOL that works with the ciliary muscles to allow people to see over a range of distances. An AIOL may be made of material(s) that imitate the optical and mechanical properties of a healthy, young lens. A fillable AIOL comprises a lens body in the form of a hollow shell which is expanded with a filling material. More recently an “all-in-one” AIOL comprising a soft, flexible material that serves as the lens body itself and does not require filling of a shell, is being developed. The AIOL is surgically inserted into the capsular bag of the eye during cataract surgery, after the cataractous lens is removed.

Existing publications describe the relative lack of both anterior capsule opacification (ACO) and posterior capsule opacification (PCO) formation in an IOL that maintains an expanded capsular bag by keeping the anterior capsule at a distance from the anterior optic surface (Kohl JC, Werner L, Ford JR, et al. Long-term Uveal And Capsular Biocompatibility Of A New Accommodating Intraocular Lens. J Cataract Refract Surg 2014; 40:2113-2119; Bontu S, Werner L, Kennedy S, et al, Long-term Uveal and Capsular Biocompatibility Of A New Fluid-Filled, Modular Accommodating Intraocular Lens. J Cataract Refract Surg 2021; 47:111-117). Such studies have shown that fibrous metaplasia of residual Lens Epithelial Cells (LECs) occurs when there is contact between the IOL optic surface and the inner surface of the anterior capsule. It is thought that by minimizing this contact and bathing the inner bag compartment in aqueous humor, LEC growth is inhibited, and capsule opacification is less likely to occur. Continuous irrigation of aqueous flow is believed to have an inhibitory effect on the proliferation of LECs due to the presence of substances in the aqueous humor such as cytokines and transforming growth factors.

Accordingly, a need exists for an accommodating intraocular lens that enables greater fluid flow about the lens body.

A need exists for an accommodating intraocular lens that reduces contact between the IOL optic surface and the inner surface of the capsular bag.

A further need exists for an accommodating intraocular lens that inhibits Lens Epithelial Cell growth between the IOL optic surface and the inner surface of the capsular bag.

### SUMMARY

A biomimetic accommodating intraocular lens (AIOL), configured to provide an amount of accommodation to allow a continuous range of vision, includes channel features to control fluid flow around the periphery of the lens. The channel geometry may vary to improve flow and may have a longitudinal profile of constant or varying depth. The number of channels may range from a single channel to numerous continuous channels around the circumference of the lens. The location of the channel(s) on the lens body may range from the edge of the lens optic zone to approximately 2-4 mm away from the optic zone towards the equator of the lens.

In addition, the channels allow for a smaller volume IOL which is easier to compress through a smaller limbal incision. The channels further allow for egress of OVD during the irrigation/aspiration step of cataract surgery.

The disclosed channels are an improvement upon an existing AIOL because the channels allow for egress of OVD during the irrigation/aspiration step of cataract surgery and further allow the eye's aqueous humor to bathe the capsular bag, thereby decreasing the incidence of anterior and posterior capsule opacification.

In accordance with one aspect of the disclosure, an accommodating intraocular lens device comprises: a deformable lens body having an exterior surface; and at least one channel extending at least partially along the exterior surface of the lens body. In embodiments, a plurality of channels extend at least partially along the exterior surface of the lens body. In embodiments, the lens body defines an equator along the exterior surface and at least one channel is disposed proximate the equator to allow fluid to flow about the lens body. In embodiments, the at least one channel has a longitudinal profile of constant or varying depth. In embodiments, the lens body defines an optic zone about a central point in the lens body wherein the channel(s) are disposed at a position ranging from approximately an edge of the anterior side optic zone to approximately 2 to 4 mm away from the optic zone towards the equator of the lens body, across the equator of the lens body, and towards the edge of the posterior side optic zone. In embodiments, the at least one channel has a cross-sectional profile having any one of an arcuate, partially rectangular, partially trapezoidal or partially T-shaped profile.

In accordance with another aspect of the disclosure, an accommodating intraocular lens device comprises: a pliable lens body having an exterior surface defining a first side and a second side and an equator region therebetween; and at least one channel in the exterior surface transiting the equator region. In embodiments, a plurality of channels transit the equator region of the exterior surface. In embodiments, the one or more channels as described herein, may extend from approximately the edge of the anterior optic zone, across the equator of the lens body, to approximately the edge of the posterior optic zone. In embodiments, the at least one channel allows fluid to flow from the first side to the second side of the lens body. In embodiments, the at least one channel has a longitudinal profile of constant or varying depth. In embodiments, at least one channel has a cross-sectional profile having any one of an arcuate, partially rectangular, partially trapezoidal or partially T-shaped profile.

In accordance with still another aspect of the disclosure, an accommodating intraocular lens device comprises: a deformable lens body having an exterior surface configuration when the lens body is in an undeformed condition; and at least one channel extending at least partially along the exterior surface of the lens body, wherein the at least one channel allows for greater deformation of the lens body such that, when compressed, the deformed lens body is insertable through a smaller incision into the eye.

In embodiments, the at least one channel has a longitudinal profile of constant or varying depth. In embodiments, at least one channel has a cross-sectional profile having any one of an arcuate, partially rectangular, partially trapezoidal or partially T-shaped profile. In embodiments, a plurality of channels extend at least partially along the exterior surface of the lens body.

In accordance with another aspect of the disclosure, a method of enabling fluid flow about an implanted accommodating lens comprises: A) acquiring an accommodating intraocular lens (AIOL) comprising a deformable lens body having an exterior surface; and at least one channel extending at least partially along the exterior surface of the lens body; and B) implanting the acquired AIOL lens in the eye of a subject in need thereof so as to allow fluid flow about the exterior surface of the lens body via the at least one channel.

### BRIEF DESCRIPTION OF THE DRAWINGS

Those skilled in the art should more fully appreciate advantages of various embodiments are described with reference to the drawings summarized immediately below.

FIG. 1 illustrates conceptually a front view of an embodiment of a 4-channel accommodating intraocular lens according to the disclosure.

FIG. 2 illustrates conceptually an isometric view of the lens of FIG. 1 according to the disclosure.

FIG. 3 illustrates conceptually a rear view of the lens of FIG. 1 with channel and optic zone dimensions according to the disclosure.

FIG. 4 illustrates conceptually a side view of the lens of FIG. 1 with channel and optic zone dimensions according to the disclosure.

FIGS. 5A-F illustrate cross-sectional views of various channel geometries to improve flow of fluid around the equator of an accommodating intraocular lens according to the disclosure.

FIG. 6 is a table of typical attributes of channels in an accommodating intraocular lens according to the disclosure.

FIG. 7 illustrates an embodiment of a 6-channel accommodating intraocular lens according to the disclosure.

FIG. 8 illustrates an embodiment of an 18-channel accommodating intraocular lens according to according to the disclosure.

FIG. 9 illustrates examples of channel dimensions of a 2-channel, 4-channel, and 18-channel accommodating intraocular lens according to the disclosure.

FIG. 10 is a photograph of a 2-channel accommodating intraocular lens according to the disclosure.

FIG. 11 is a photograph of a 4-channel accommodating intraocular lens according to the disclosure.

FIG. 12 is a photograph of a 6-channel accommodating intraocular lens according to the disclosure.

FIG. 13 is a photograph of a 18-channel accommodating intraocular lens according to the disclosure.

### DETAILED DESCRIPTION

The present disclosure will be more completely understood through the following description, which should be read in conjunction with the drawings. In this description, like numbers refer to similar elements within various embodiments of the present disclosure. The skilled artisan will readily appreciate that the methods, apparatus and systems described herein are merely exemplary and that variations can be made without departing from the spirit and scope of the disclosure.

The purpose of an accommodating intraocular lens (AIOL) is to restore the ability of the human eye to accommodate under typical visual stimuli. Applicant's prior U.S. Pat. Nos. 10,278,810 and 11,678,976 both describe fluid filled, accommodating IOLs comprising a capsular shell or interface enclosing an optically acceptable medium. The device establishes a physiologic range of optical power in response to a range of ciliary contractile states. The medium provides shape to the capsular interface, optical power, and a physiologic response to the suspensory ligament. The capsular shell or interface of such fluid filled IOLs may be modified to include the features described herein that enable greater fluid flow about the body of the disclosed accommodating intraocular lens.

International Publication WO2023225332A1 describes polymers that can be used to make an all-in-one AIOL that does not require a shell.

Disclosed herein is an AIOL including features to control fluid flow around the periphery of a biomimetic AIOL. In accordance with the disclosure, an accommodating intraocular lens includes one or more channels extending between the anterior and posterior optic zones and transiting the equator region of the lens body to allow fluid such as Ophthalmic Viscoelastic Device (OVD) to be flushed out from behind the lens and to allow fluid like aqueous humor to continuously bathe the AIOL. Advantages of the channel design include prevention of OVD retention and inhibition of posterior capsule opacification (PCO).

In embodiments, a biomimetic accommodating intraocular lens (AIOL) could include a shell and a liquid filler disposed within the shell. The shell has a modulus and a refractive index. The liquid filler has a viscosity and a refractive index. A biomimetic AIOL could also include one material that can be molded into an all-in-one AIOL that requires no shell. The intraocular lens is configured to provide an amount of accommodation to allow a continuous range of vision. Either type of AIOL could include features to control fluid flow around the periphery of the lens.

The number of channels may range from a single channel to numerous continuous channels around the circumference of the lens. The location of the channel(s) on the lens could range from the edge of the optic zone to approximately 2-4 mm away from the optic zone towards the equator of the lens. The width of each channel could range from approximately 25 microns to 2 mm, and the depth of each channel could range from 25 microns to 2 mm. The channel longitudinal profile could be a constant depth or varying depth of channel geometry. The channel geometry may vary to improve flow.

In accordance with one embodiment, an accommodating intraocular lens includes a shell, and a liquid filler disposed within the shell. In accordance with another embodiment, an AIOL consists of an all-in-one material. Either AIOL could include grooves, or channels, that allow for egress of Ophthalmic Viscoelastic Device (OVD) during the irrigation/aspiration procedure of cataract surgery. The channels extend from the anterior portion of the lens to the posterior portion of the lens. Both the anterior and posterior channels could begin outside of the central optical zone. The channels will allow Balanced Salt Solution (BSS) to flow from the anterior to the posterior region of the capsular bag during the irrigation/aspiration (I/A) step of surgery, flushing out the OVD by allowing the OVD to flow from the posterior to the anterior region of the capsular bag where it will be aspirated using an I/A device.

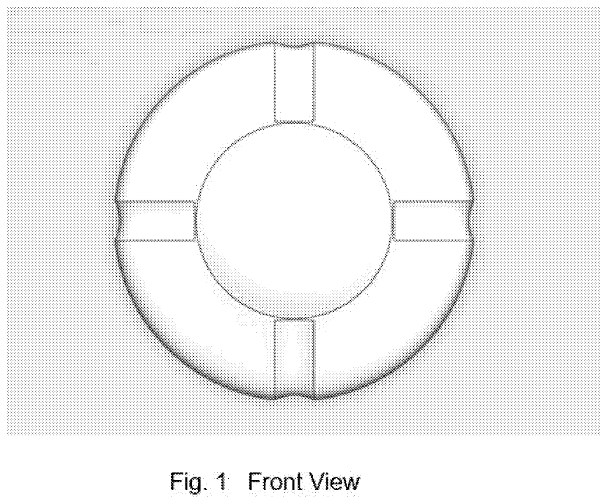

FIGS. 1-4 show conceptually front, isometric, and rear views of an accommodating intraocular lens according to an illustrative embodiment of the disclosure. In embodiments, the AIOL comprises a deformable/compressible lens body having an exterior surface and a channel or groove extending at least partially along the exterior surface of the lens body.

In embodiments, the lens body itself may comprise a shell or interface, as described in U.S. Pat. Nos. 10,278,810 and 11,678,976, filled with the fluid mediums disclosed therein or those disclosed in US Patent Application Publication 2024/0287232A1 or International Publication WO2024/233709A2.

In embodiments, the lens body itself may alternatively comprise an integrally formed flexible body made of a biocompatible material, as described in WO2023/225332A1.

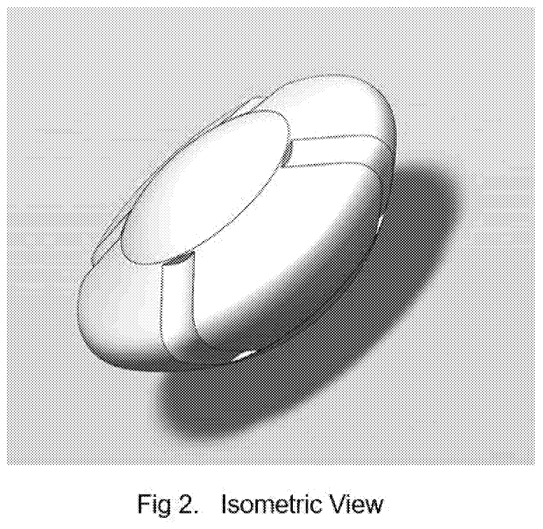

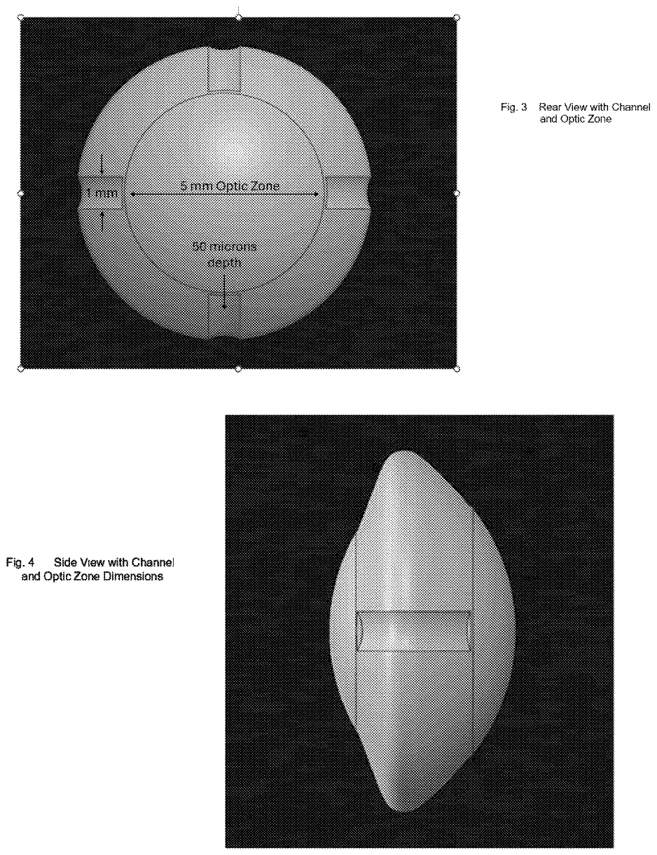

In embodiments, the lens body is substantially disc shaped with a generally circular exterior perimeter and an enlarged thickness central area on both sides thereof, as illustrated in the top view of FIG. 1 and rear view of FIG. 3. The enlarged thickness central area on both the sides of the lens body defines a substantially round optic zone extending from a first side of the lens body through to a second side of the lens body, the optic zone being delineated in FIGS. 2-4. In embodiments, the optic zone on the rear side of the lens body may be approximately 5 mm in diameter, as shown in FIG. 3, but may range anywhere from 3 mm to 6 mm in diameter. The optic zone at the front side of the lens may be the same or different than on the rear side.

The generally circular exterior perimeter of the lens body defines a curved transition region or equator intermediate the front and rear side optic zones of the lens body, as illustrated in the isometric view of FIG. 1 and side view of FIG. 3. In embodiments, the equatorial diameter of the lens body may be 9-11 mm.

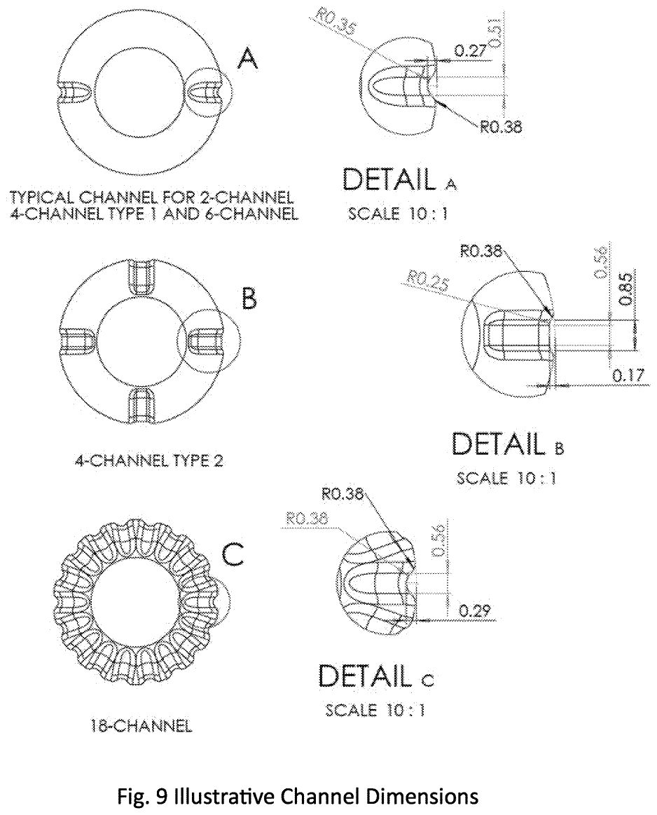

In embodiments, one or more channels or grooves extend along the exterior surface of the lens body, as illustrated in FIG. 1-4 and 7-9. In embodiments, the lens body may have any number of channels, e.g. 2, 4, 6, 8, etc., extending at least partially along the exterior surface of the lens body. Exemplary dimensions of the channels being shown in FIGS. 6 and 9.

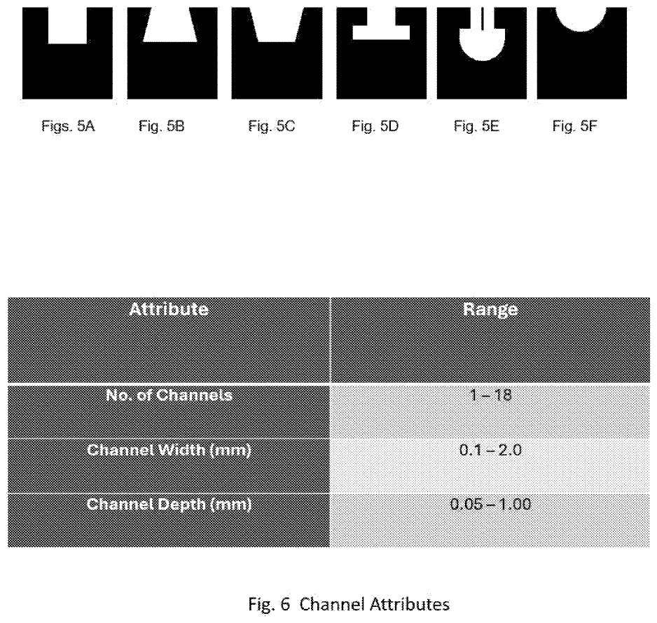

FIGS. 5A-F illustrate cross-sectional views of various channel geometries to improve flow of fluid around the equator of the lens body. FIG. 5F illustrates a partial cross-sectional view of a lens body in which a channel has an arcuate cross-sectional profile similar to those illustrated in FIGS. 1-4 and 7-9. FIG. 5E is a variation of the channel profile illustrated in FIG. 5F. FIG. 5A illustrates a partial cross-sectional view of a lens body in which a channel has a square or partially rectangular cross-sectional profile. FIG. 5B illustrates a partial cross-sectional view of a lens body in which a channel has a partially trapezoidal cross-sectional profile. FIG. 5C illustrates a partial cross-sectional view of a lens body in which a channel has a partially T-shaped cross-sectional profile.

In any of the embodiments illustrated in FIGS. 5A-F, one or a plurality of channels extend at least partially along the exterior surface of the lens body and may have the same or different or various combination cross sectional profiles. In any of the embodiments illustrated in FIGS. 5A-F, one or a plurality of channels may have longitudinal profile of constant or varying depth along the length thereof.

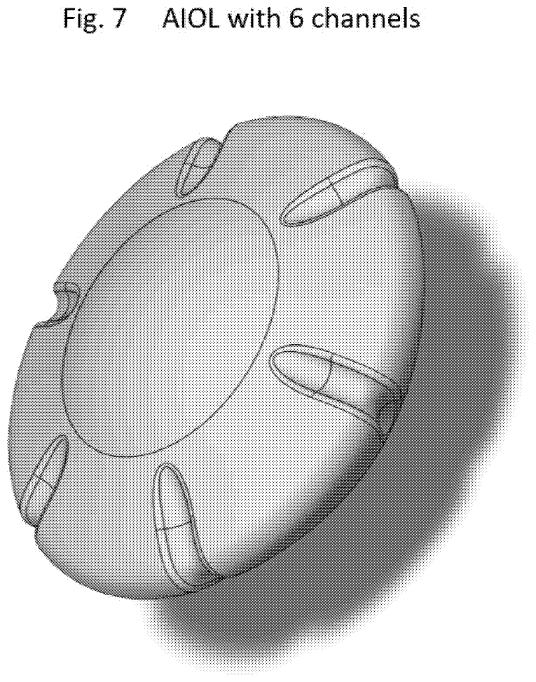

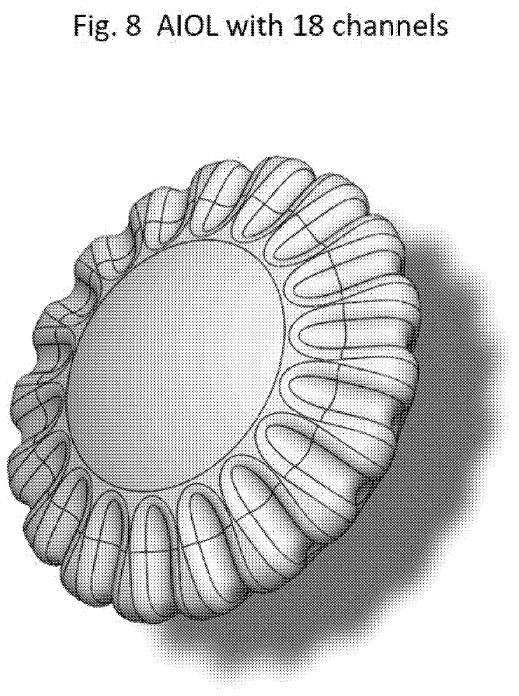

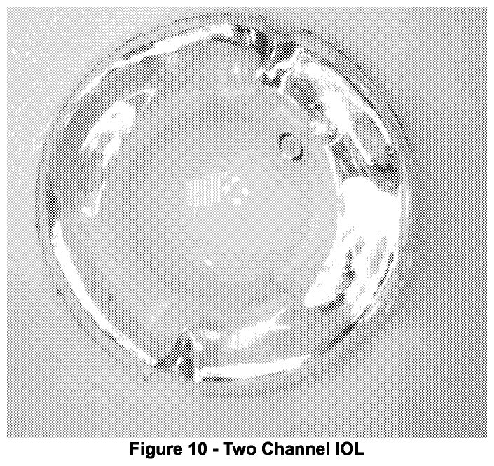

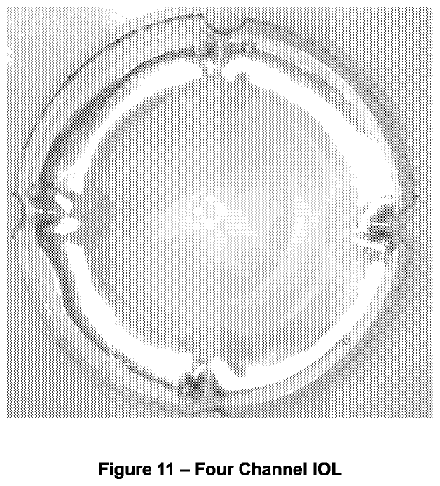

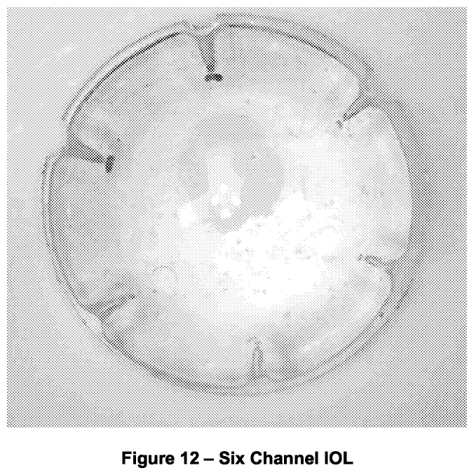

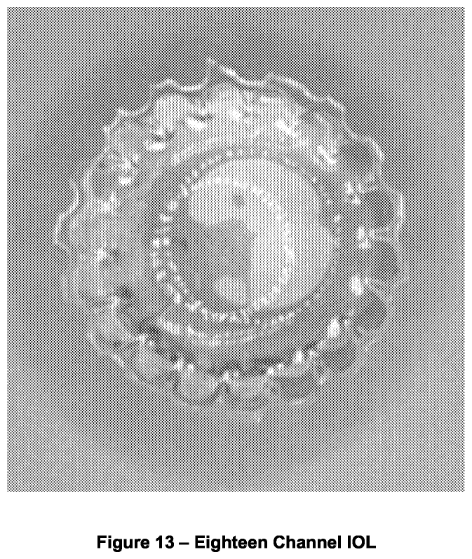

FIG. 6 illustrates a table of exemplary channel attributes and attribute value ranges, including no. of channels, channel width, and channel depth, of in an accommodating intraocular lens according to the disclosure. FIG. 9 further illustrates the proximity of 2-channel, 4-channel, and 18-channel configurations to a lens body optic zone and exemplary dimensions of each channel configuration, including the channel width and depth at the equator region of the lens body. The channel configurations disclosed in FIG. 9 improve flow of fluid around the equator of the lens body while maintaining a stable optic zone of the accommodating intraocular lens. FIGS. 7-8 illustrate 6-channel and 18-channel configurations of an accommodating intraocular lens according to the disclosure, including the relationship of the channels to the equator region and optic zones of the lens body. FIGS. 10-13 are photographs of a 2-, 4-, 6- and 18-channel accommodating intraocular lens according to the disclosure, respectively.

The various AIOL channel configurations disclosed herein allow the eye's aqueous humor to bathe the inner surface of the eye's capsular bag, thereby decreasing posterior capsule opacification formation. Further, an open and expanded capsular bag with aqueous humor flow helps to decrease the incidence of anterior capsule opacification.

In accordance with another aspect of the disclosure, the disclosed channels allow the lens to function like the young, healthy, human lens, but with a smaller overall volume. The disclosed AIOL, with one or more channels in the exterior surface of the lens body, occupies less space in the injector or other delivery mechanism, making it easier to get through a smaller limbal incision and into the eye. In practice, an undeformed AIOL with at least one exterior channel has uncompressed volume and shape profile. Upon deformation thereof, the total volume of the lens body remains the same but the shape profile becomes smaller, due to the presence of one or more channels in the exterior surface of the lens body, allowing for easier insertion into a limbal incision and into the capsular bag of the eye. In this manner the one or more channels also facilitate a reduction of the overall space occupied by AIOL during surgical deployment for easier insertion into the capsular bag of the eye.

In accordance with another aspect of the disclosure, a method of enabling fluid flow about an implanted accommodating lens comprises: A) acquiring an accommodating intraocular lens (AIOL) comprising a deformable lens body having an exterior surface and at least one channel extending at least partially along the exterior surface of the lens body; and B) implanting the acquired AIOL lens in the eye of a subject in need thereof so as to allow fluid flow about the exterior surface of the lens body from a first side of the lens body to a second side of the lens body via the at least one channel.

In practice the channels may be any of etched, machined or molded into the exterior surface of the shell of a fillable lens body or directly into the lens body of a pre-formed AIOL. Although the channel configurations disclosed herein have been described with reference to an AIOL device such channel configurations are also applicable to other lens devices as well including IOL devices.

At various places in the present specification, values are disclosed in groups or in ranges. It is specifically intended that the description includes each and every individual sub-combination of the members of such groups and ranges and any combination of the various endpoints of such groups or ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

For purposes of clarity and a concise description, features are described herein as part of the same or separate embodiments, however, it will be appreciated that scope of the concepts may include embodiments having combinations of all or some of the features described herein.

It will be apparent to those recently skilled in the art that modifications to the apparatus and process disclosed here in may occur, including substitution of various component values or nodes of connection, without parting from the true spirit and scope of the disclosure as defined by the claims set forth herein.

What is claimed is:

1. An accommodating intraocular lens device, comprising:
a deformable lens body having an exterior surface; and
at least one channel extending at least partially along the exterior surface of the lens body.

2. The device of claim 1, comprising a plurality of channels extending at least partially along the exterior surface of the lens body.

3. The device of claim 2, wherein the plurality of channels ranges from 2 to 18 channels.

4. The device of claim 1, wherein the lens body defines an equator along the exterior surface thereof and wherein the at least one channel is disposed proximate the equator to allow fluid to flow about the lens body.

5. The device of claims 1, wherein the at least one channel has a cross-sectional profile optimized to improve fluid flow.

6. The device of claim 1, wherein the at least one channel has a longitudinal profile of constant depth or wherein the at least one channel has a longitudinal profile of varying depth.

7. The device of claim 1, wherein the at least one channel has a width dimension ranging from approximately 25 microns to greater than 1 mm.

8. The device of claim 1, wherein the at least one channel has a depth dimension ranging from approximately 25 microns to greater than 1 mm.

9. The device of claim 4, wherein the lens body defines an optic zone having a size dimension ranging from approximately 3 mm from a central point on the exterior surface of the lens body to approximately 6 mm from the central point on the exterior surface of the lens body.

10. The device of claim 9, wherein the plurality of channels extend outwardly approximately 2 mm to approximately 4 mm from the optic zone towards the equator of the lens body.

11. The device of claim 1, wherein the at least one channel has an arcuate shaped cross-sectional profile or wherein the at least the one channel has a partially rectangular shaped cross-sectional profile or wherein the at least one channel has a partially trapezoidal shaped cross-sectional profile or wherein the at least one channel has a partially T-shaped cross-sectional profile.

12. An accommodating intraocular lens device, comprising:
a pliable lens body having an exterior surface defining a first side and a second side and an equator region therebetween; and
at least one channel in the exterior surface transiting the equator region.

13. The device of claim 12, comprising a plurality of channels in the exterior surface transiting the equator region.

14. The device of claim 12, wherein the at least one channel allows fluid to flow from the first side to the second side of the lens body.

15. The device of claim 12, wherein the at least one channel has a longitudinal profile of constant depth or wherein the at least one channel has a longitudinal profile of varying depth.

16. The device of claim 12, wherein the at least one channel has an arcuate shaped cross-sectional profile or wherein the at least the one channel has a partially rectangular shaped cross-sectional profile or wherein the at least one channel has a partially trapezoidal shaped cross-sectional profile or wherein the at least one channel has a partially T-shaped cross-sectional profile.

17. An accommodating intraocular lens device, comprising:
a deformable lens body having an exterior surface configuration when the lens body is in an undeformed condition; and
at least one channel extending at least partially along the exterior surface of the lens body,
wherein the at least one channel allows for a deformation of the volume of the lens body such that, when compressed, the deformed volume of the lens body is insertable through a smaller incision into the eye.

18. The device of claim 17, comprising a plurality of channels extending at least partially along the exterior surface of the lens body.

19. The device of claim 17, wherein the at least one channel allows fluid to flow from a first side to a second side of the lens body.

20. The device of claim 17, wherein the at least one channel has a longitudinal profile of constant depth or wherein the at least one channel has a longitudinal profile of varying depth.

21. The device of claim 17, wherein the at least one channel has an arcuate shaped cross-sectional profile or wherein the at least the one channel has a partially rectangular shaped cross-sectional profile or wherein the at least one channel has a partially trapezoidal shaped cross-sectional profile or wherein the at least one channel has a partially T-shaped cross-sectional profile.

22. A method of enabling fluid flow about an implanted accommodating lens, comprising:
A) acquiring an accommodating intraocular lens (AIOL) comprising a deformable lens body having an exterior surface and at least one channel extending at least partially along the exterior surface of the lens body; and
B) implanting the acquired AIOL in the eye of a subject in need thereof so as to allow fluid flow about the exterior surface of the lens body from a first side of the lens body to a second side of the lens body via at least one channel.
